(19) United States
(12) Patent Application Publication
(10) Pub. No.: US 2024/0417965 A1
(43) Pub. Date: Dec. 19, 2024

(54) SHOWER DRAIN DEVICES, SHOWER DRAIN SYSTEMS, AND METHODS OF MANUFACTURING AND USING THE SAME

(71) Applicant: KHUONG KIM BACH, HO CHI MINH (VN)

(72) Inventor: KHUONG KIM BACH, HO CHI MINH (VN)

(21) Appl. No.: 18/159,086

(22) Filed: Jan. 24, 2023

(30) Foreign Application Priority Data
Aug. 3, 2022 (VN) 1-2022-04928
Aug. 3, 2022 (VN) 1-2022-04929

(51) Int. Cl. E03F 5/04; G01F 23/00; G01N 33/00

(52) U.S. Cl. CPC E03F 5/0408; G01F 23/0007; G01N 33/0044; G01N 33/0063

(57) ABSTRACT

A shower drain and a method for draining off excess water from a floor to a sewer are provided which includes: a water inlet unit configured to receive the excess water; a double layer water outlet unit, mechanically coupled to the water inlet module to form a reservoir having a bottom connecting chambers that contain a body of water at a predetermined height that force the excess water beyond said predetermined height to overflow into the sewer; and a water level sensor circuitry placed inside double layer water outlet unit for transmitting the water level information inside the reservoir.

### CLAIMS OF PRIORITY

This application claims priority under 35 U.S.C. § 119 (a) of applications No. 1-2022-04928 and No. 1-2022-04929, entitled “ [symbol] thoát nu′ó′c và [symbol] giám sát ngăn mùi và [symbol] thoát nu′ò′c này”, filed on Aug. 3, 2022 in the Republic Socialist of Vietnam. The patent applications identified above are incorporated here by reference in their entirety to provide continuity of disclosure.

### FIELD OF THE INVENTION

The present invention relates generally to shower drains. More specifically, the present invention relates to a shower drain having dual connecting chambers overflowing mechanism designed to keep out bad odor and to minimize clogging.

### BACKGROUND ART

All bathrooms have shower drains on the shower floors to drain off water. Shower drains—connected to drainpipes which lead water to the sewer—comprise a hollow cylindrical plastic body portion and a metal cover grate or strainer. The drains are positioned on the pipe so that the cover grate is flush with the shower floors. Often shower drains are often clogged by hair and debrise build-up such as soaps, dirts, or grimes. The clogged drains are more than just a nuisance. They can cause flooding or sewer backup. When this issue is allowed to progress, contaminated water floods all over the floor giving off rotten egg smell. Finally, expensive water damage to the bathroom results. Typically, the rotten egg smell is created when bacteria grows in the drainage system and breaks down waste, forming hydrogen sulfide (H₂S).

There are many attempts to solve the shower drain clogging problems. One of the methods has been the use of a p-trap which is a U-shaped or P-shaped section of the drainage system positioned right after the drain. P-trap holds water which acts as a barrier to prevent gas in the drainage system from seeping back up the drain into the home. However, if the P-trap is dry, then there is not any water in the trap to block the gas from seeping up through the drain. This happens if the bathroom has not been used for a longtime or blocked by solid waste or broken drainage vent. If the bathroom has not been used for a while, then water has to be used to fill up the P-trap. But when there is a blocked or broken drainage vent, a vacuum in the drain line is created, which empties the water in the P-trap. In addition, dirt and grime that has been collected in the bottom of the P-trap may also be causing the rotten egg smell because this area is prone to bacteria growth.

Next referring to FIG. 1, a prior-art shower drain 100 that uses overflowing or spill-over mechanism to block out rotten egg odor is illustrated. Shower drain 100 is a goose neck (G-type) that uses overflowing mechanism to retain water at the G-neck section. This water blocks bad odor H₂S gases from the sewer. Shower drain 100 includes a strainer body 101, a locknut 102, and a trap section 103. Trap section 103 includes an inner filter 106 and two openings 104 and 105. Inner filter 106 is in a form of a mesh intended to retain hair and solid wastes while passes water 111 to trap section 103. As water 121 rises to the rims of openings 104 and 105, it overflows outward to the sewer. Shower drain 100 is usually made with materials coated with chrome (Cr), Nickel (Ni). Customers have commented that the prior art shower drain 100 or the likes are slow in draining off water due to its structure that is difficult to integrate to the bath floor. Most importantly, bad odors are still seeping up toward the bath floor because hair and solid soap residues are easily intertwined and clogging inner filter 106.

In high-rise buildings or condominium projects, when bathrooms in higher floors are clogged, it is often difficult to remove the clogging debrises from shower drain 100 in consideration to residents on the floor below. In higher floors apartments, clogs tend to clump together deep into the pipes due to gravity. When flooding, clogged drains, and leaky water sources caused damage to the apartments, it is difficult to repair the damaged walls caused by water. Therefore, it is essential in high-rise condominium projects to use shower drains to avoid clogs, floods, and water damage.

When people turn on the water faucet in a bathroom or bathtub. They often forget to turn it off due to some distractions such as telephone calls, an ongoing TV program or a movie that they are watching, etc. This causes flooding and water seeping to the floors below. Consequently, precious water is wasted. Ceiling, floor, and walls of the residences below are also damaged. Shower drain 100 of the prior-art fails to fix these problems. The markets and consumers such as elderlys people need a solution to these above described problems.

Therefore, what is needed is a new shower drain that can retain a body of water that blocks bad odor gases such as hydrogen sulfide (H₂S) and carbon monoxide (CO), etc.

What is needed is a new shower drain that can effectively drain off water on the bath floor.

What is needed is a new shower drain that can be easily to install or remove from the bath floors.

Furthermore, what is needed is new shower drains that are clog free that can be used in high rise buildings and condos.

Finally, what is needed is a shower drain that can be both decorous and effective in draining waste water.

The first type (surface drain) and second type (edge drain) shower drains and the method of manufacturing the same disclosed in the present invention solve the above-described problems and objectives.

### SUMMARY OF THE INVENTION

Accordingly, an object of the present invention is to provide a shower drain and a method for draining off excess water from a floor to a sewer are provided which includes: a water inlet unit configured to receive the excess water; a double layer water outlet unit, mechanically coupled to the water inlet module to form a reservoir having a bottom connecting chambers that contain a body of water at a predetermined height that force the excess water beyond said predetermined height to overflow into the sewer; and a water level sensor circuitry placed inside double layer water outlet unit for transmitting the water level information inside the reservoir.

Another object of the present invention is to provide a method for making a shower drain for draining off excess water from a floor to a sewer which includes the following steps: (a) providing a water inlet unit configured to receive the excess water; and (b) providing a double layer water outlet unit, mechanically coupled to the water inlet unit to form a reservoir with bottom connecting chambers that force the excess water beyond a predetermined height to overflow into a sewer, and (c) detecting the water level inside the reservoir by placing a water level sensor circuit coupled to an antenna on top of the water inlet unit.

Another object of the present invention is to provide a shower drain system that includes water level sensor configured to sense the water levels within the connecting chambers. The water sensor is installed inside the shower drains transmitting water level and flooding signals to a gateway nearby. The gateway includes receivers for receiving the water levels and flooding signals as well as odor, temperature, light, and image signals. A server communicates with the gateways to transmit the signals to a central control room so that water damage and inconveniences can be avoided and/or prevented.

Yet another object of the present invention is to provide a network

Another object of the present invention is to provide shower drain that can retain a body of water that blocks bad odor gases such as hydrogen sulfide (H₂S).

Another object of the present invention is to provide a new shower drain that can effectively drain off water on the bath floor.

Another object of the present invention is to provide a new shower drain that can be easily to install or remove from the bath floors.

Another object of the present invention is to provide new shower drains that are clog free or resistant to clog which can be used in high rise buildings and condos.

Another object of the present invention is to provide a shower drain that can be both decorous and conveniently placed anywhere on the bathroom floor.

These and other advantages of the present invention will no doubt become obvious to those of ordinary skill in the art after having read the following detailed description of the preferred embodiments, which are illustrated in the various drawing figures.

### BRIEF DESCRIPTION OF THE DRAWINGS

The accompanying drawings, which are incorporated in and form a part of this specification, illustrate embodiments of the invention and, together with the description, serve to explain the principles of the invention.

FIG. 1 shows a prior-art bath strain that employs the overflowing of water that blocks bad odors;

FIG. 2 shows a three-dimension (3D) perspective diagram of a first type shower drain (surface drain) in accordance with an exemplary embodiment of the present invention;

FIG. 3A shows a pattern that is both decorous and effective in draining off water of the first type shower drain in accordance with a first exemplary embodiment of the present invention;

FIG. 3B shows another pattern among many patterns that is both decorous and effective in draining off water of the first type shower drain in accordance with a first exemplary embodiment of the present invention;

FIG. 4 shows three-dimension (3D) diagram of the first structure of the first type shower drain in accordance with an aspect of the present invention;

FIG. 5 shows three-dimension (3D) cut-away diagram of the first structure of the first type shower drain when separated into top part and bottom part in accordance with an aspect of the present invention;

FIG. 6 shows three-dimension (3D) diagram of the first structure of the first type shower drain when combined the top part and the bottom part together in accordance with an aspect of the present invention;

FIG. 7 shows the two-dimension (2D) diagram that illustrates the operating principle of the first type shower drain in accordance with an aspect of the present invention;

FIG. 8 shows the three-dimension (3D) diagram of a second type shower drain (edge flow) in accordance with an aspect of the present invention;

FIG. 9 shows the three-dimension (3D) diagram illustrating the structure of the second type shower drain (edge flow) in accordance with an aspect of the present invention;

FIG. 10 shows the two-dimension (2D) diagram illustrating the inner structure of the second type shower drain (edge flow) in accordance with an aspect of the present invention in accordance with an aspect of the present invention;

FIG. 11 shows the two-dimension (2D) diagram that illustrates a second-type bath drain system and its operating principle in accordance with an aspect of the present invention;

FIG. 12 is a schematic diagram of a wireless water level sensor transmitter module in accordance with an aspect of the present invention;

FIG. 13 is a schematic diagram of a wireless gateway module receiver module in accordance with an aspect of the present invention in accordance with an aspect of the present invention;

FIG. 14 shows a schematic diagram of a bath drain system capable of detecting water level and flood in a bathroom floor of a house in accordance with an exemplary embodiment of the present invention;

FIG. 15 shows a system level of a server for the bath drain system capable of detecting water level and flood in a bathroom floor in accordance with an exemplary embodiment of the present invention;

FIG. 16 is a network of bath drain systems that connect gateway modules and servers together in accordance with an exemplary embodiment of the present invention;

FIG. 17 is a flow chart of a method of manufacturing the bath drain system including electronic sensors and in accordance with an aspect of the present invention; and

FIG. 18 shows a flow chard of a method of use of bath drain system including detecting water level and unintentional bathroom flood in accordance with an aspect of the present invention.

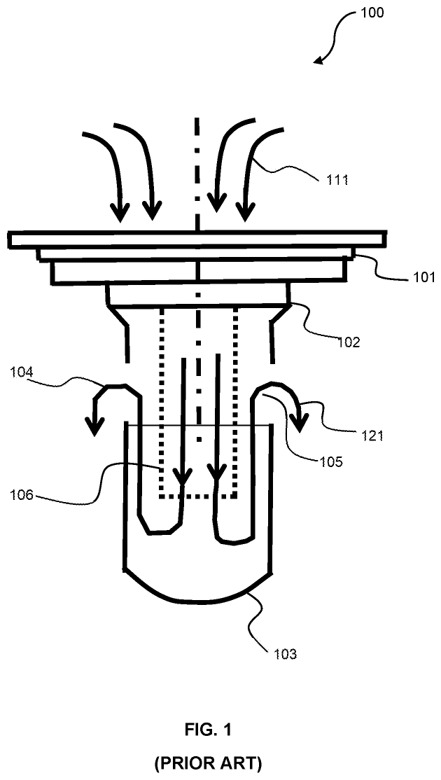

The figures depict various embodiments of the technology for the purposes of illustration only. A person of ordinary skill in the art will readily recognize from the following discussion that alternative embodiments of the structures and methods illustrated herein may be employed without departing from the principles of the technology described herein.

### DETAILED DESCRIPTION OF THE INVENTION

Reference will now be made in detail to the preferred embodiments of the invention, examples of which are illustrated in the accompanying drawings. While the invention will be described in conjunction with the preferred embodiments, it will be understood that they are not intended to limit the invention to these embodiments. On the contrary, the invention is intended to cover alternatives, modifications and equivalents, which may be included within the spirit and scope of the invention as defined by the appended claims. Furthermore, in the following detailed description of the present invention, numerous specific details are set forth in order to provide a thorough understanding of the present invention. However, it will be obvious to one of ordinary skill in the art that the present invention may be practiced without these specific details. In other instances, well-known methods, procedures, and components have not been described in detail so as not to unnecessarily obscure aspects of the present invention.

Disclosed in the present specification is novel bath showers that include a water inlet unit removably inserted to a water outlet unit whose operation is based on the bottom connecting chambers principle to force water and debrises into the sewer. In many preferred embodiments of the present invention, a water level sensor and other sensors such as odor sensor are provided that detect the water levels inside the water outlet unit. The odor sensor and other sensors are conveniently packaged in a gateway module that is wall mounted outside of the shower drains to detect the hydrogen sulfide (H₂S) and other gases such as carbon monoxide (CO) inside the bathroom. Yet in many embodiments, the shower drains and the sensors are networked using gateway modules and servers to monitor the water flow inside the bathrooms in order to avoid flooding and clogging situations, thus saving water and repairing costs. The network of bath drains of the present invention are applicable in smart cities, condominium projects, hotels, resorts, apartment complexes.

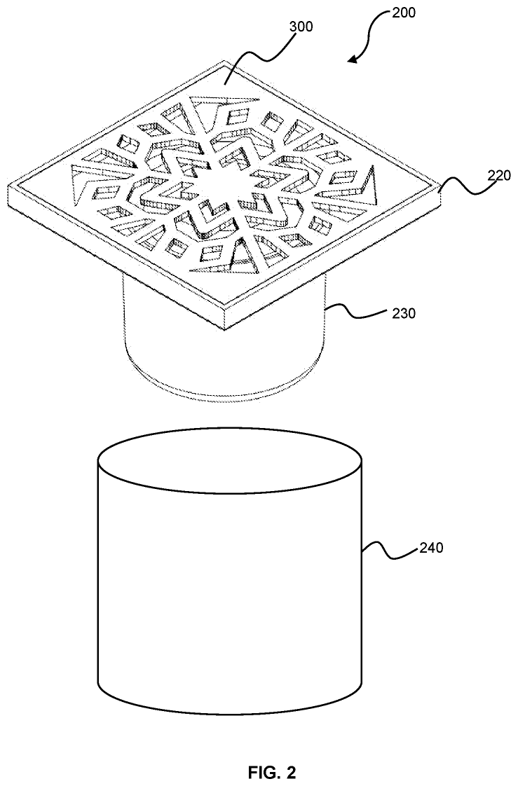

Now referring to FIG. 2, a 3-dimensional (3D) perspective diagram of a first type (surface drain) shower drain 200 in accordance with an exemplary embodiment of the present invention is illustrated. First type shower drain 200 is a surface drain that allows water to drain off from various copyrightable patterns imprinted on the surface of a lid 300. Next is a panel 220 connected to a double layer outer core 230 which is plugged into a tailpiece 240. Tailpiece 240 is an opening that leads water to the sewer. In first type shower drain 200, shower water (not shown) enters the openings cut into various patterns on lid 300 and then falls into double outer core 230 and then to tailpiece 240 and finally to the sewer system. Panel 220 and double layer core 230 are easy to separate. They are also easily removed from tailpiece 240. Double layer core 230 has connecting chambers that (a) keep a fixed water column and (b) force overflowing (spill-over) of any excess water into tailpiece 240 so that the water level is in equilibrium in both chambers. This fixed amount of water columns blocks bad odor gases such as hydrogen sulfide (H₂S) or carbon monoxide (CO). After removed, panel 220 and double layer outer core 230 can be cleared from hair and solid wastes such as soaps, grimes, or dirts.

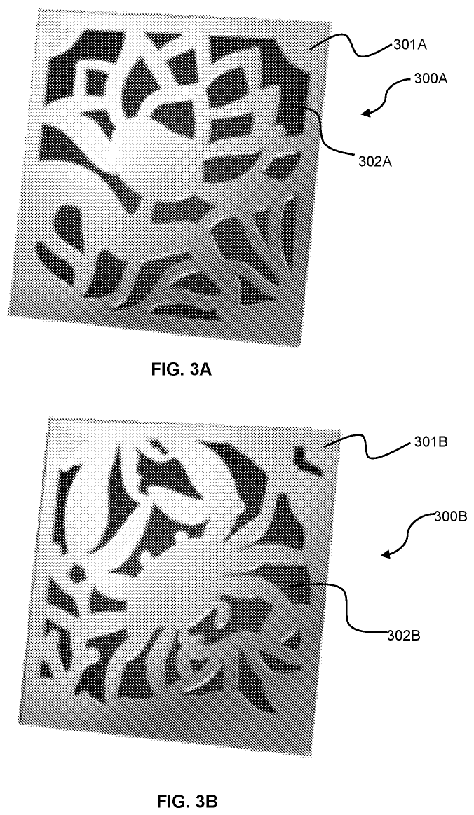

In FIG. 3A, a lotus flower pattern cut on lid 300A in accordance with an exemplary embodiment of the first type shower drain 200 is illustrated. Lid 300A has a solid background 301A and openings 302A that are patterned into the lotus flower shape. The openings 302A and the structure of panel and double layer outer core 230 are designed to ensure high drainage speed. In many embodiments of the present invention, the drainage speed of first type shower drain 200 is 25-200 liters per hour. Lotus flower patter 300A can be accomplished by laser cutters, 3D printers, or CNC machine tools.

Next in FIG. 3B, a crab pattern cut lid 300B in accordance with an exemplary embodiment of first type shower drain 200 is illustrated. Crab pattern 300B has a solid background 301B and openings 302B that are patterned into the crab shape. The openings 302A and the structure of panel and double layer outer core 230 ensure high drainage speed. In many embodiments of the present invention, the drainage speed of first type shower drain 200 is 25-200 liters per hour. Crab pattern 300B can be accomplished by laser cutters, 3D printers, or CNC machine tools. It is noted that other copyrightable patterns of lid 300 are within the scope of the first type shower drain 200.

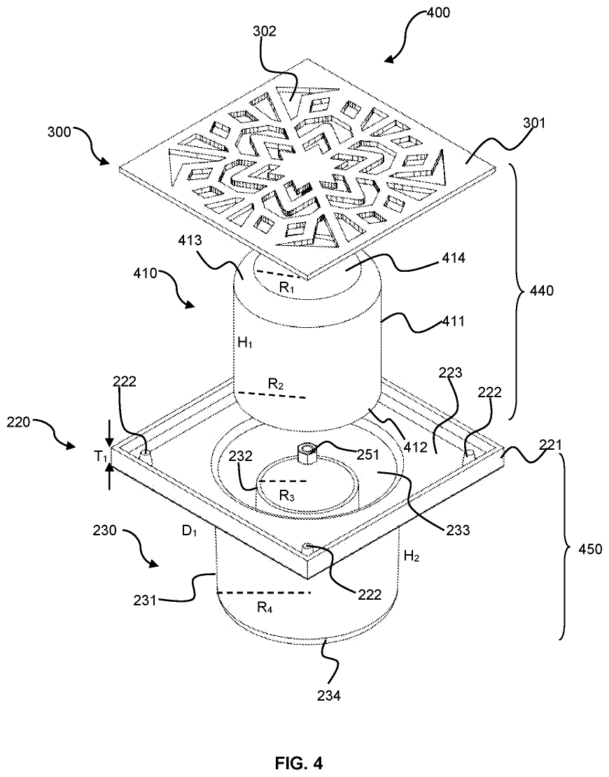

Next referring to FIG. 4, a three-dimension (3D) diagram showing the structure of a first type shower drain 400 in accordance with an exemplary embodiment of the present invention is illustrated. Shower drain 400 includes lid 300 removably coupled to an inner core 410 by a lock nut 251. In many embodiments of the present invention, inner core 410 includes a top circular surface 414 with a first radius R₁ connected to a truncated cone section 413. Truncated cone section 143 is extended into a cylindrical body section having a first height H₁ and a second radius R₂. Top circular surface 414 of inner core 410 is a solid surface while the bottom surface 412 of cylindrical body section 411 is opened (hollow). Lid 300 and inner core 410 constitutes a water inlet module 440 of shower drain 400.

Continuing with FIG. 4, panel 220 is an open top cubic box with lateral side 221 having a thickness D₁ and height T₁. In various embodiments of the present invention, thickness T₁ is 2-10 mm and height T₁ is 10-80 mm. Double layer outer core 230 is situated at the center of panel 220. Double layer outer core 230 has the shape of open top truncated cone with a truncated cone inner layer 232 with radius R₃ and a truncated cone outer layer 231 with radius R₄. Double layer outer core 230 is designed that R₄ is greater than R₃. With that, a reservoir 233 exists between inner layer 232 and outer layer 231. The base of panel 220 is inclined at an angle θ between 20° to 30° toward the center so that excess water flows toward reservoir 233. At the four corner of panel 220, there are four spacers 222 having a height about 5 mm to 20 mm. Panel 220 is fused with double layer outer 230 to form a water outlet unit 450. In assembly, lid 300 is connected to inner core 410 by lock nut 251 to form water inlet unit 440. Then, water inlet unit 440 is removably coupled to water outlet unit 450. In various embodiments of the present invention, shower drain 400 is made up stainless steel or Inox 304 which contains 8.1% Nickel (Ni), 1% manganese (Mn), 18% Chromium (Cr), and 72.9% iron (Fe).

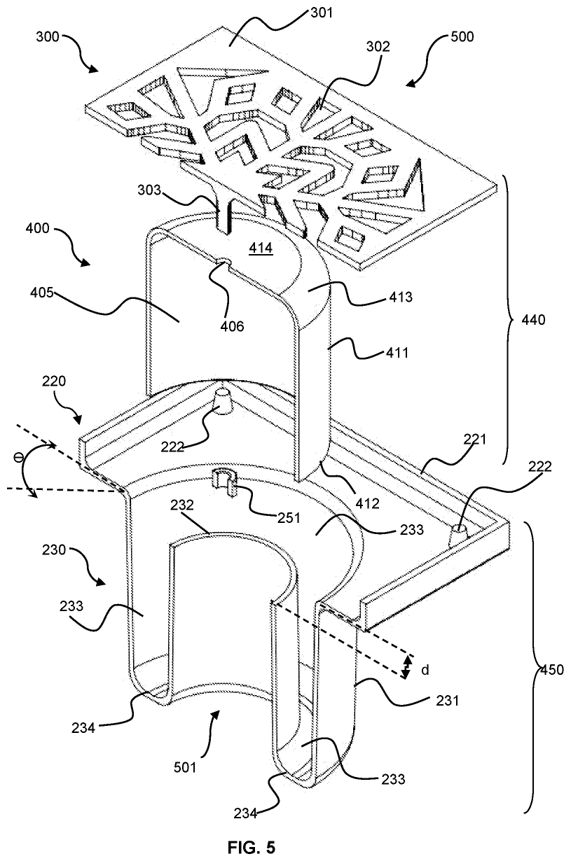

Now turning to FIG. 5, a 3D cutaway diagram of that shows the inner structure shower drain 200 in accordance with an exemplary embodiment of the present invention is illustrated. As seen, lid 300 includes a threaded bolt 303 protruding downward for mating with lock nut 251. Upper surface 414 of inner core 410 includes a thru hole 406 for threaded bolt 303 to pass through. Inner core 410 is hollow with a hollow space 405. For double layer outer core 230, inner layer 232 has the top rim lower than that of outer layer 231 by a distance d approximately 5-50 mm. Inner layer 232 is hollow with a hollow space 501. Outer layer 231 is hollow above a solid bottom side 234. This form reservoir 233.

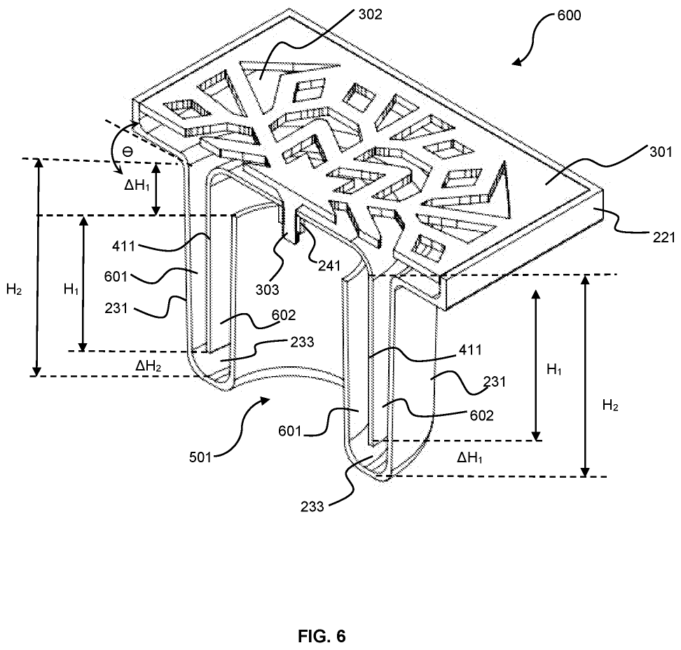

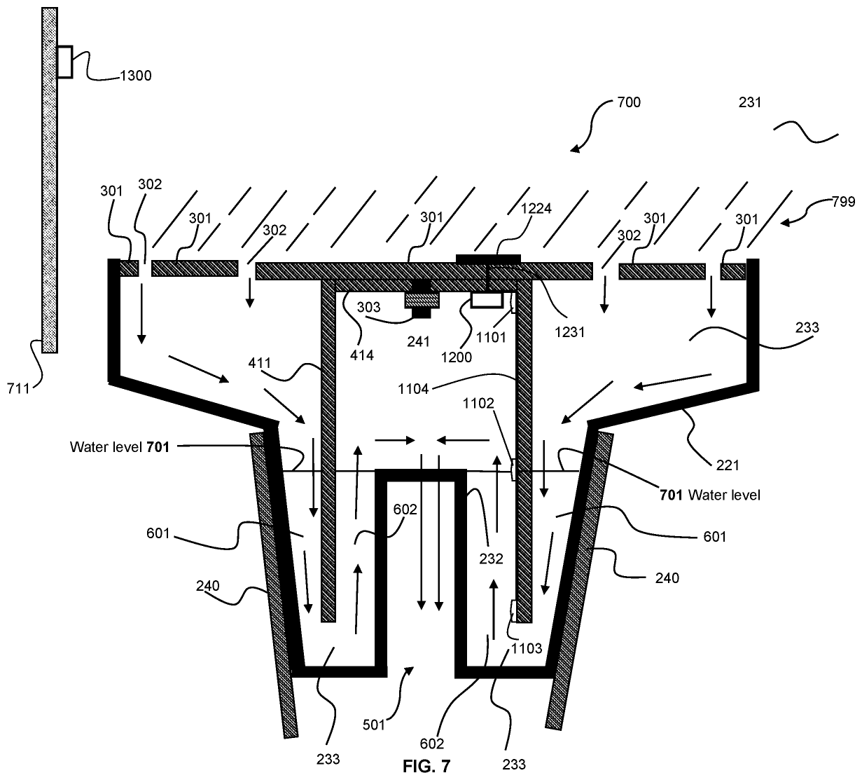

FIG. 6 illustrates a fully assembled first type shower drain 600 in accordance with an exemplary embodiment of the present invention. When lid 300 is secured to inner core 410 with nut locknut 251, top surface of lid 300 is flush with the edge of panel 220. Inner core 410 is completely contained within outer core 230. Due to the dimensional differences between inner layer 410 and outer layer 231, i.e., height ΔH₁ at the top and ΔH₂ at the bottom and radius R₃ and R₄, a first chamber 601 and a second chamber 602 are formed inside hollow space 233. In many preferred embodiments of the presentation invention, H₁ is 3-120 mm, ΔH₁ is 4-50 mm, and ΔH₂ is 4-50 mm. The operations of first type shower drain 200 is illustrated in FIG. 7.

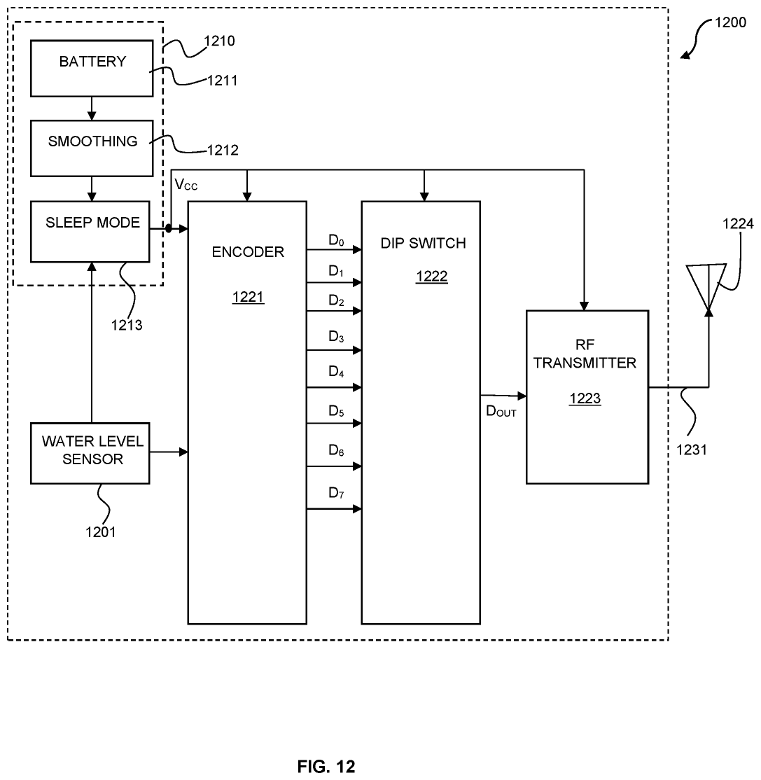

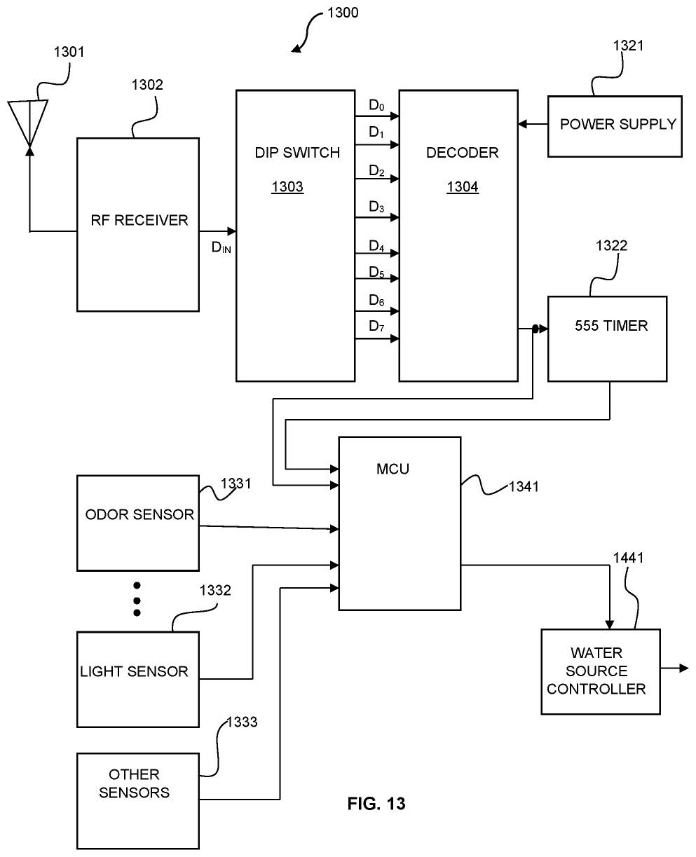

Now referring to FIG. 7, a 2D diagram representing the first type shower drain system 700 including an odor sensor and a water level sensor in combination with other sensors in accordance with an exemplary aspect of the present invention is illustrated. In many embodiments of the present invention, an odor sensor 1331 fabricated inside a gateway module 1300. Gateway module 1300 is mounted outside of shower drain 200 for detecting bad odor such as hydrogen sulfide (H₂S) emanating from the sewer. In some embodiments of the present invention, gateway module 1300 is mounted on a wall or on the ceiling 711 of a bathroom. Gateway module 1300 also contains other types of sensors that will describe more in FIG. 13. Advert now to FIG. 7, Hydrogen sulfide is a colorless gas with is toxic, corrosive, and flammable. This triple threat, combined with high toxicity at low levels make early detection essential. This H₂S gas is produced through bacteria breakdown of organic matter, making its presence relatively easy within confined spaces of the sewer. Human nose can smell the characteristic rotten egg smell of H₂S from around 0.5 ppm. Odor sensor 1331 is Alphasense H₂S sensor. In other embodiments, carbon monoxide and H₂S sensor such as COH-A2 sensor is used. The structure and operation of odor sensor 1331 are well known in the art and need not discussed in details within the present application. A water level sensor system 1200 is attached to the bottom side of top surface 414 of cylindrical section 411 of inner core 400. An antenna 1224, electrically coupled to water level sensor system 1200 via an electrical cable 1231, is placed outside atop of solid background 301 to transmit the water level information to gateway module 1300. Water levels are sensed by a first (high) metal probe 1101, a second (middle) metal probe 1102, and a third (low) metal probe 1103, all connected together by an electrical cable 1104. In various preferred embodiments of the present invention, water level sensor system 1200 detects both water level within hollow space 233, first chamber 601, and second chamber 602. As a consequence, water level detector 1200 can detect unintentional flood caused by distracted and forgetful owners. If high water level is maintained for Tₘᵢₙ of 30 minutes or more, water level sensor system 1200 inform the owner and concierges, and then turn off the water source. The schematic and operation of water level sensor 1200 is described in full in FIG. 12.

In operation, after water 711 from the shower or from other sources such as faucets is flowing through openings 302 of lid 300 filling up first chamber 601 and second chamber 602. The water level is eventually raises to the rim of inner core 401 and overflow to the sewer via space 501. Hair and assortment of solid wastes such as soaps, dirts, and slimes, etc. are collected at the bottoms of first chamber 601 and second chamber 602. Due to the principle of connecting chambers, water levels on both sides of first chamber 601 and second chamber 602 are forced to be the same. This creates a force to continuously push clump of hair and debrises into second chamber 602. Thus, first type shower drain 200 of the present invention is clog free. In rare occasions when first chamber 601 and second chamber 602 were clogged, owner and/or concierge are informed. In this case, lid 300 and inner core 231 are easily removed by a hook (not shown) through openings 302 to expose panel 220 and hollow space 233 of first chamber 601 and second chamber 602. Clogs are easily removed. Water level sensor system 1200 is for extra security when shower drain 200 is both clogged and water kept running. With the design of first type shower drain 200, even when owner forgot to turn off the water source, the connecting first chamber 601 and second channel 602 are always forcing water to be flown into hollow space 501 then to the sewer. This is due to the connecting first chamber 601 and second chamber 602 cause a water level 701 in both chambers to be the same. In many embodiments of the present invention, the height of water column inside first chamber 601 and second chamber 602 is 3-120 mm and the drainage speed is 25-200 liters per hour. The operations of water level sensor system 1200 and gateway module 1300 will be described in FIG. 12 and FIG. 13 respectively.

FIG. 2 to FIG. 7, first type shower drain 200 achieves the following objectives of the present invention:
(1) high drainage speed;
(2) clog free;
(3) easy to assemble;
(4) aesthetically decorous in any bathrooms
(5) easy to clean;
(6) dangerous gas detection; and
(7) flood detection

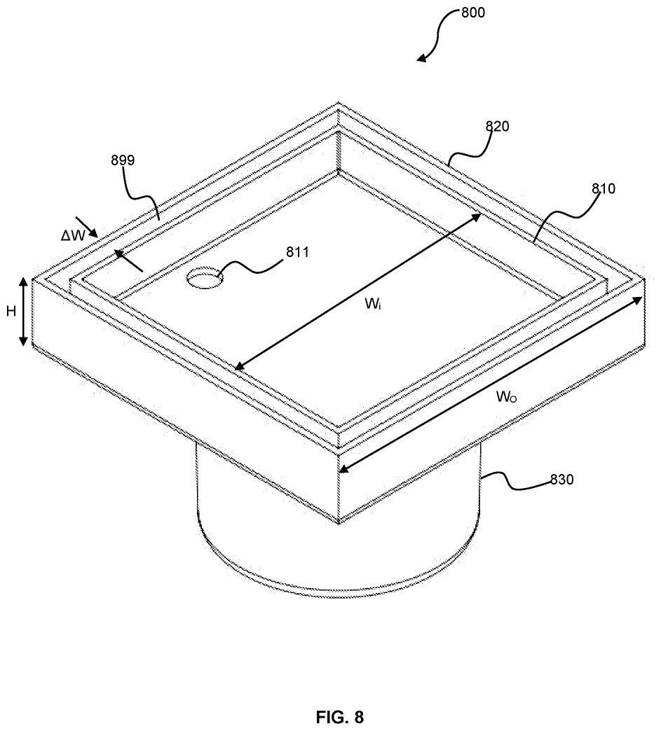

FIG. 8 to FIG. 11 describe a second type shower drain of the present invention that is edge drain. The second type shower drain of the present invention operates the same principle as first type shower drain 200 described in FIG. 2-FIG. 7. The operating principle of both first type shower drain and second type shower drain is the overflowing of connecting double chamber. The only difference is the drainage or the manner water is drained.

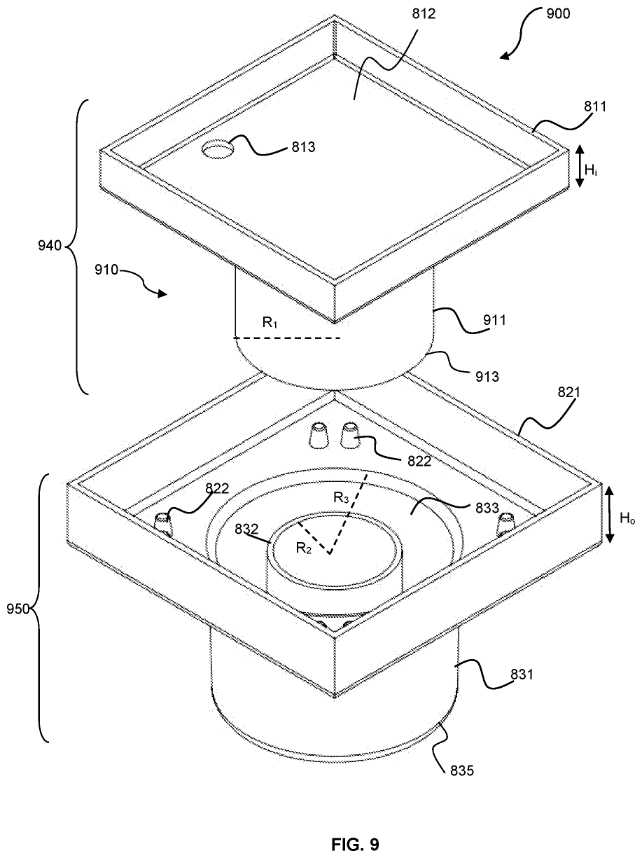

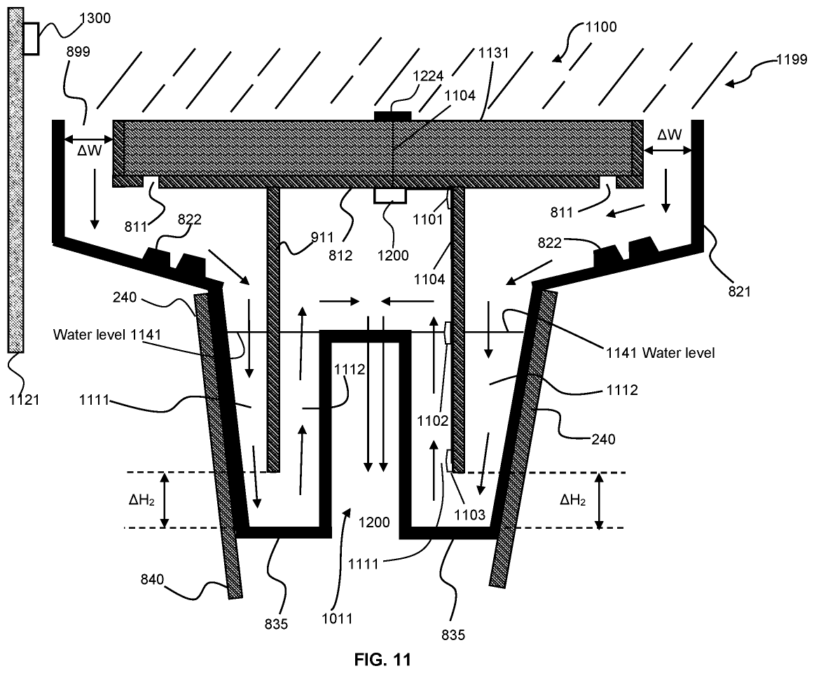

Now referring to FIG. 8, a three dimension (3D) perspective diagram of a second type shower drain 800 that uses edge drain mechanism in accordance with an exemplary embodiment of the present invention is illustrated. Shower drain 800 includes an inner panel 810, an outer panel 820, an inner core 910 (see FIG. 9), and a double layer outer core 840. Double layer outer core 840 is inserted snuggly to tailpiece 240 (see FIG. 11) of the sewer system (not shown). Inner panel 810 and outer panel 820 have the same open top parallelepiped shape whose base is a square. The width Wᵢ of inner panel 810 is slightly smaller than the width Wₒ of outer panel 820. The thickness T of both inner panel 810 and outer panel 820 are the same and is 2-10 mm. As seen, inner panel 810 is placed inside outer panel 820 so that a gutter drainage 899 of width ΔW is formed along the common perimeter of both inner and outer panels 810 and 820. The height H of both inner panel 810 and outer panel 820 is the same and substantially less than their widths Wᵢ and Wₒ. The height of inner panel 810 and outer panel 820 is 15-80 mm. Therefore their rims are flushed to one another and to the floor of the bathroom (not shown). On the top surface of the base of inner panel 810, there are holes 811 designed for ejecting floor bricks (not shown). A hollow space 813 of inner panel 810 is designed to hold a decorative floor brick.

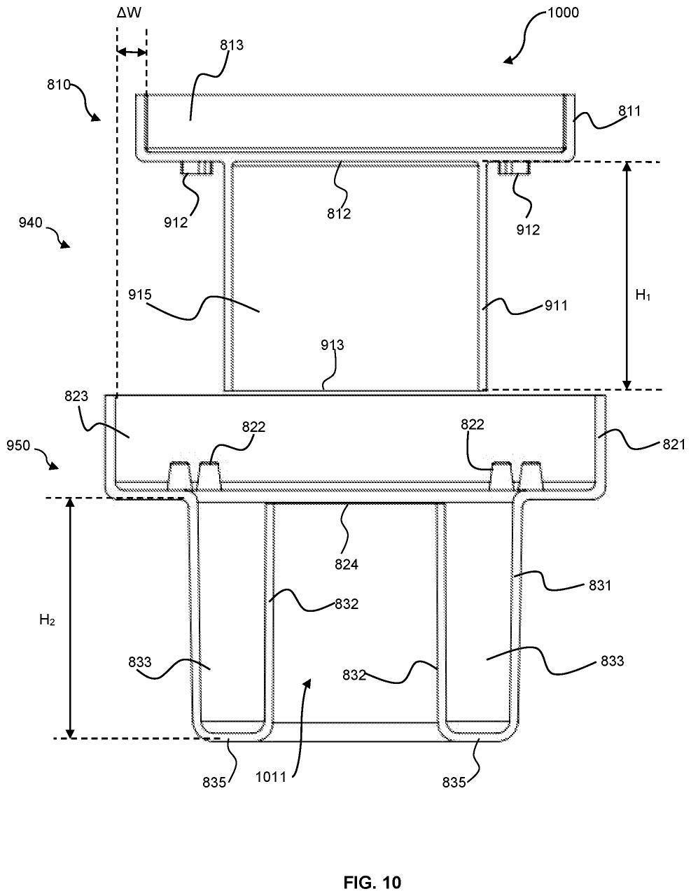

Next, referring to FIG. 9, a three dimension (3D) perspective diagram of a second type shower drain 800 before assembling in accordance with an exemplary embodiment of the present invention is illustrated. As seen, inner panel 810 is connected with inner core 910 to form a water inlet unit 940. Inner core 910 is shaped as a hollow truncated cone with a lateral side 911 and an open bottom 913. In many embodiments of the present invention, inner core 910 includes a truncated cone section with a radius R₁ and lateral side 911. In the second type shower drain embodiment, inner core 910 is fused permanently at the center of a bottom side 812 of inner panel 810 to form water inlet unit 940. The structure of outer panel 820 is the same as that of shower drain 400 described above in FIG. 4 except that outer panel 820 has more spacers 822 designed to support the weight of decorative floor bricks and those of users. Double layer outer core 830 has the shape of an open top truncated cone and is fused to second panel 820 to form a water outlet unit 950. Double layer outer core 830 includes an outer layer 831 with radius R₃ concentric with an inner layer 832 with radius R₂, where R₃ is greater than R₂. With this design, a reservoir 823 is formed between inner layer 832 and outer layer 831. The bottom surface of outer panel 820 is inclined at an angle θ between 20° to 30° toward the center so that excess water flows toward hollow space 823. The flowing rate or drainage speed is 25 to 200 liters per hour. At the four corner of outer panel 820, there are eight spacers 822 having a height about 5 mm to 20 mm. To assemble, inner core 910 with truncated cone 911 are inserted into double layer outer core 830 until bottom side 812 of inner panel 810 rests firmly on spacers 822. FIG. 10 will further disclose the inner structure of shower drain 800.

Referring now to FIG. 10, a 2D diagram of that shows the inner structure shower drain 800 in accordance with an exemplary embodiment of the present invention is illustrated. As seen, inner panel 810 is a hollow open top parallelepiped with an inner hollow space 813. At bottom side 812 there are supporting foots 912. Inner core 910 includes a lateral side 911 and a hollow inner space 912 and an open bottom side 913. Lateral side 911 has a height H₁. Inner panel 810 is fused permanently to inner core 910 to form water inlet unit 940. The interior side of outer panel 820 is planted with spacers 822 on both sides. Spacers 822 and feet 812 are designed to support the total weight on shower drain 800 since second type of shower drain 800 may be installed in the middle of the bathroom. Decorative bath brick installed inside the hollow space 813 are the same as those in the bathroom. While first type shower drain 200 is usually placed at the corners or near the walls, outer panel 820 is also a open top hollow parallelepiped 821 with an inner hollow space 823. A bottom side 824 of second open top hollow parallelepiped 821 is fused permanently with double layer outer core 830 to form water outlet unit 950. Double layer outer core 830 is an open top hollow truncated cone with outer layer 831 and inner layer 832, both with height H₂. Inner layer 832 has a radius R₂ which is smaller than the radius R₃ of and outer layer 831. Henceforth, a reservoir 833 is formed therebetween. Since inner layer 832 is an open top hollow truncated cone, it has a hollow space 1011 in fluid communication with the sewer.

Now referring to FIG. 11, a 2D diagram of a second type shower drain system 1100 in accordance with an exemplary aspect of the present invention is illustrated. In many embodiments of the present invention, an odor sensor 1331 fabricated inside a gateway module 1300. Gateway module 1300 is mounted outside of shower drain 800 for detecting bad odor such as hydrogen sulfide (H₂S) emanating from the sewer. In some embodiments of the present invention, gateway module 1300 is mounted on a wall or on the ceiling 1121 of a bathroom. Gateway module 1300 also contains other types of sensors that will describe more in FIG. 13. Odor sensor 1331 is Alphasense H₂S sensor. In other embodiments, carbon monoxide and H₂S sensor such as COH-A2 sensor is used. The structure and operation of odor sensor 1331 are well known in the art and need not discussed in details within the present application. A water level sensor system 1200 is attached to the bottom side 812 of inner panel 810. An antenna 1224, electrically coupled to water level sensor system 1200 via an electrical cable 1231, is placed outside atop of a decorative brick 1131 to transmit the water level information to gateway module 1300. Water levels are sensed by a first (high) metal probe 1101, a second (middle) metal probe 1102, and a third (low) metal probe 1103, all connected together by an electrical cable 1104. In various preferred embodiments of the present invention, water level sensor system 1200 detects both water level within reservoir 833, first chamber 1111, and second chamber 1112. As a consequence, water level detector 1200 can detect unintentional flood caused by distracted and forgetful owners. If high water level is maintained for Tₘᵢₙ of 30 minutes or more, water level sensor system 1200 inform the owner and concierges, and then turn off the water source. The schematic and operation of water level sensor 1200 is described in full in FIG. 12.

Water 1131 from the shower or from other sources such as faucets are flowing through edges 1101 formed between first panel 810 and second panel 820, filling up first chamber 1111, second chamber 1112, third chamber 1113, and fourth chamber 1114. The water level eventually raises to the top rim of inner core 401 and overflows to the sewer via space 1011. Hair and assortment of solid wastes such as soaps, etc. are collected at the bottoms of first chamber 1111 and second chamber 1112. Due to the principle of connecting tanks, water levels 1141 on both sides of first chamber 1111 and second chamber 1112 are always the same. This creates a force to continuously push hair and debrises into second chamber 1112 and eventually to hollow space 1011 leading to the sewer. Thus, second type shower drain 800 of the present invention is clog free. In rare occasions when reservoir 833 were clogged, first panel 810 is easily removed by a hook, a grasp, or a thong (not shown) through edge 1101 to expose reservoir 833 for clean-up. Clogs are easily removed.

Continuing with FIG. 11, water level sensor system 1200 is for extra security when shower drain 800 is both clogged and water was allowed to run. With the design of second type shower drain 800, even when owner forgot to turn off the water source, the connecting first chamber 1111 and second channel 1112 are always forcing water to be flown into hollow space 1011 then to the sewer. This is due to the connecting first chamber 1111 and second chamber 1112 cause a water level 701 in both chambers to be the same. In many embodiments of the present invention, the height of water column inside first chamber 1111 and second chamber 1112 is 3-120 mm and the drainage speed is 25-200 liters per hour. The operations of water level sensor system 1200 and gateway module 1300 will be described in FIG. 12 and FIG. 13 respectively.

FIG. 8 to FIG. 11, second type shower drain 800 achieves the following objectives of the present invention:
(1) high drainage speed;
(2) clog free;
(3) easy to assemble;
(4) aesthetically decorous in any bathrooms
(4) easy to clean; and
(5) can be decorative that matches the pattern of the floor bricks.

Now turning to FIG. 12, a schematic diagram of a water level sensor system 1200 that is placed inside either first type shower drain 700 or second type of shower drain 1100 in accordance with an exemplary embodiment of the present invention is illustrated. Please advert to FIG. 7 and FIG. 11 for the placements of water level sensor system 1200 inside each type of shower drain 200 or 800. Water level sensor system 1200 is a wireless transmitter that transmits the water level inside connecting chambers 1111 and 1112. Water level sensor system 1200 includes a water level sensor integrated circuit (IC) 1201 that outputs various digital signal levels to an encoder 1221, and a DIP switch 1222. The output of DIP switch 1222 is fed to a wireless transmitter 1223 that uses antenna 1224 (see FIG. 7 and FIG. 11). A power supply unit 1210 provides a voltage supply V_{CC} to the above listed components. Power supply unit 1210 is battery operated which includes a 9-V battery, a smoothing circuit 1212, and a sleep mode circuit 1213.

In many embodiments of the present invention, water level sensor 1201 is a ULN2003 which are connected to a high metal probe 1101, a medium metal probe 1102, and a low metal probe 1103 for detecting high, middle, and low water levels respectively. The electrical connections of ULN2003 for water level detection are shown in the datasheet and need not described in details herein. Encoder 1221 is an integrated circuit (IC) HT12E. The parallel outputs (D₀ to D₇) of encoder 1221 are fed into DIP switch 1222 which converts these parallel data to serial data D_{OUT}. This output data is fed to transmitter 1223. Transmitter 1223 is an ASK RF transmitter transmitting the water level signals at 433 MHZ. Sleep mode circuit 1213 is a power management integrated circuit (PMIC) LM10504 made by Texas Instruments Inc.

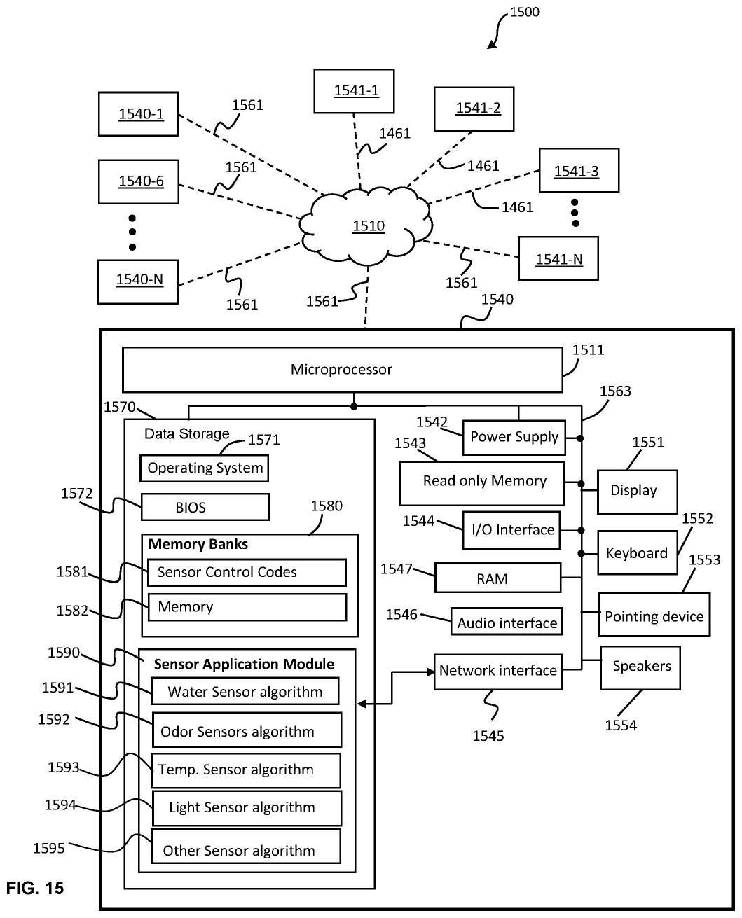

Next referring to FIG. 13, a schematic diagram of a gateway module 1300 that receives signals from either first type shower drain 700 or second type of shower drain 1100 and other sensors in accordance with an exemplary embodiment of the present invention is illustrated. Gateway module 1300 is an intermediate circuitry designed to collect all sensors signals and send them to a server for further processing (see FIG. 15). Gateway module 1300 includes an RF antenna 1301 coupled to a receiver 1302. Output signal from receiver 1302 is fed to a DIP switch 1303 and then to a decoder 1304. Outputs of decoder 1304 are fed to at least one timers 1331. Timers 1331 are 555 ICs that set time thresholds for high water level and low water level respectively. Other sensors including an odor sensor 1331, a light sensor 1332, and/or other sensors 1333 such as temperature sensor (thermometer) are connected to a microcontroller unit (MCU) 1341 for processing. MCU 1341 receives information from water level sensors 1201, timer 1322, odor sensor 1331, light sensor 1332, and other sensors 1333 (e.g., thermostat, and/or cameras). MCU 1341 processes the information and issues appropriate commands to water source controllers 1441.

In various embodiments of the present invention, antenna is 1301 a though-hole mount 433 Mhz RF antenna model CRFT-MG0433-30170-1M-SMA with 2 dB gain and 50Ω impedance. For wall-mounted gateway module 1300, receiver antenna can be either spring, FPC, PCB, rubber duck, fiberglass, through hole screw mount antenna. Decoder 1304 is an integrated circuit (IC) HT12D. The parallel outputs DIN of encoder 1221 are fed into decoder 1304 which converts water level signal data to parallel data (D₀ to D₇). Odor sensor 1331 is either a hydrogen sulfide (H₂S) and/or carbon monoxide (CO) sensor. H₂S sensor is a MQ-136 Arduino compatible. Light sensor 1332 is a BH 1750 Arduino light sensor. Temperature sensor 1333 is a DHT11, LM35DZ, or LM335 integrated circuits. Water source controller 1441 is equipped with an Arduino solenoid water valve. MCU 1341 is Arduino microcontrollers such as Node MCU (ESP8266), Teensy 3.6 that uses a 32-bit 180 MHz ARM Cortex-M4 processor, MSP430 Launchpad, the Multi-tool STM32, PocketBeagle (the Linux Alternative), or a FPGA which is programmable gate arrays. It is noted that other ICs and control language such as Linux, C++ that perform the same disclosed functions are within the scope of the present invention.

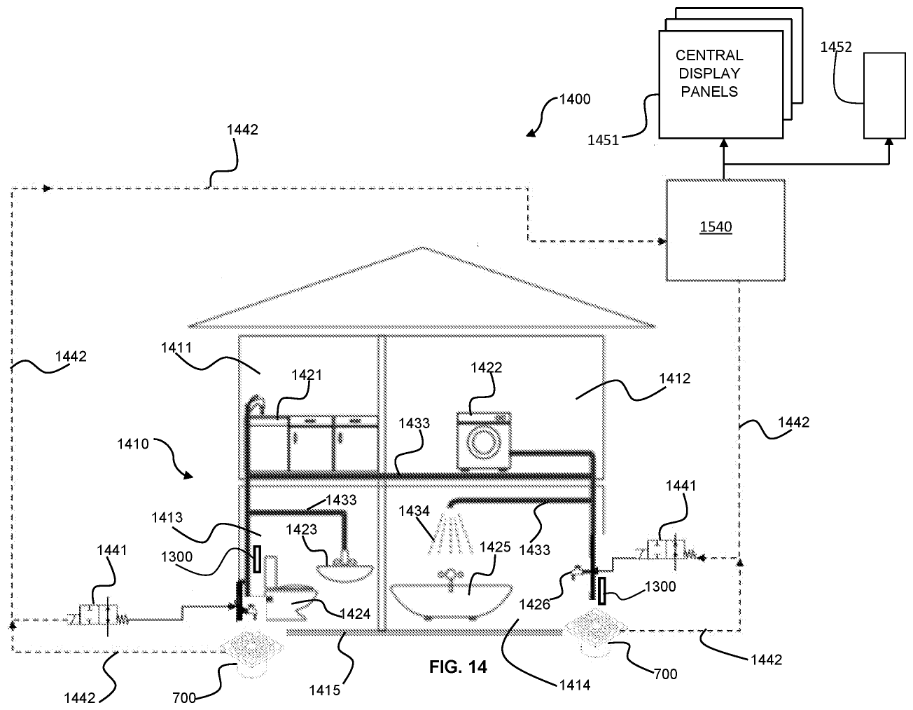

FIG. 14 is a schematic diagram of dwellings such as smart homes, offices, condos, or apartments 1400 that are equipped with the shower drain system in accordance with an exemplary embodiment of the present invention. The shower drain systems 700 and 1100 as described in FIG. 7, FIG. 11, FIG. 12, and FIG. 13 above are used to achieve the following objectives of the present invention:

(a) a clog free shower drain that uses the fluid connecting chambers to eliminate clogging.

(b) water sensor and transmitter that communicates the water level information inside the shower drain to avoid dry off, flooding, bad odor, and thus avoiding damage to the shower floor and possibly rooms there below.

(c) Users, homeowners, hotel concierges, control room employees can be notified of the drainage situation in each room, thus avoiding flood damage, bad odor, and waste of water leading to saving money and efforts.

Smart home system 1400 includes a plurality of residences 1410. In each residence 1410, there are a kitchen 1411 with at least one kitchen sinks 1421, a laundry room 1422 with a washing machine 1422, a bath room 1413 with washbasin (or sink) 1413 and a toilet bowl 1414, a shower room 1414 with a bathtub 1425. All of these fixtures are connected to water conduits 1433. Water tubes transport water 1435 to all the above listed rooms. Water 1434 and its flow rate are controlled by solenoid valve controllers 1441. Bathroom and shower room floors 1415 are installed with first type shower drains 700 for draining off excess water 1434. Water level sensor system 1200 inside each shower drain 700 communicates the water level information to gateway modules 1300 and then to a server 1540 via communication channels 1442. Server 1530 is located in a control room of a hotel, or an apartment complex (condominium, a high-rise office building). Server 1540 receives sensor information from shower drain system 700, odor sensor 1331, light sensor 1332, other sensor 1333 and, in turn, processes these information to issue appropriate commands. For example, if one of the shower drains 700 is dried (or low water level), server 1540 commands solenoid valve water faucet 1426 to release a certain amount of water until the water level reaches at least at the middle level. Alternatively, server 1540 can download the shower drain algorithms to gateway module 1300. In this situation, MCU 1341 will take action in place of server 1540. Server 1540 displays the water situation in each bathroom 1413 and shower room 1414 in house 1410 on a series of display panels 1411. At the same time, server 1540 can send alarm to a smartphone 1412 to inform each owner.

Now referring to FIG. 15, a schematic diagram of a network servers 1500 in accordance with an exemplary embodiment of the present invention is illustrated. It will be noted that a network of servers 1500 can be connected as a cluster of different servers 1540-1 to 1540-N connected and serviced by a network 1510. Network of servers 1500 also connects smartphones 1541-1, 1541-2, 1541-3, to 1541-N to network 1510. Each server 1540-1 to 1540-N collects and processes information from bathroom of a hotel, a condominium project, or a smart single resident house. Servers 1540-1 to 1540-N are linked to one another via a communication channel 1561. Server 1540 of the present invention is representative of other servers 1540-1, 1540-2, . . . 1540-N and connected to edge/fog/cloud network 1510 via communication channel 1561. In some embodiments of the present invention, all servers 1540-1, 1540-2, and 1540-N may be connected together in a master-slave configuration via communication channel 1561 and one server (e.g., 15400) can serve as a network to the other servers 15400-2 to 1540-N. As alluded above, networks 1540-1 to 1540-N can be data center, cloud/edge/fog, or network such as nanonetwork, body area network (BAN), personal area network (PAN), local area network (LAN), campus/corporate area network (CAN), metropolitan area network (MAN), wide area network (WAN), and mesh area networks, or any combinations thereof. Communication channels 1561 can be wireless channels such as Bluetooth, 4G, LTE, 5G, Wi-Fi, Zigbee, Z-wave, radio frequency (RF), Near Field Communication (NFC), Ethernet, LoRaWAN, or can be wired connectors such as RS-232, RS-485, USB, or any combinations thereof. Server of the present invention 1540-1, 1540-2 and 1540-N may use each other as a network using different communication protocols such as Message Queue Telemetry Transport (MQTT), Data Distribution Service (DDS), HTTP, TCP/IP, (Advanced Message Queuing Protocol (AMQP), Modbus, BACnet, OPCUA, or any combinations thereof. Servers 1540-1 to 1540-N may be set up by different manufacturers having different physical connections, communication protocols, industrial standards, as well as operating parameters. They can communicate to one another via communication channel 1561 as long as networks 15401-1 to 1540-N can be linked together, i.e., via a common network such as an edge/fog/cloud network 1510. Alternatively, these servers, 1540-1 to 1540-N may use one another as an intermediary server to communicate to other servers outside of cloud network 1510.

Continuing with FIG. 15, a typical server 1540 of the present invention includes a microprocessor 1511 in communication with a memory 1470 via a bus 1563. Server 1540 also includes a power supply 1542, a network interface 1545, a Read Only Memory (ROM) 1543, Random Access Memory (RAM) 1547, a local input/output interface 1544, a display 1551, a keyboard 1552, audio interface 1554, and a pointing device driver 1553. Power supply 1542 provides necessary voltages supplies to server 1540. Local input/output interface 1544 enables input and output communication between microprocessor 1511, display 1551, keyboard 1552, and pointing device driver 1553.

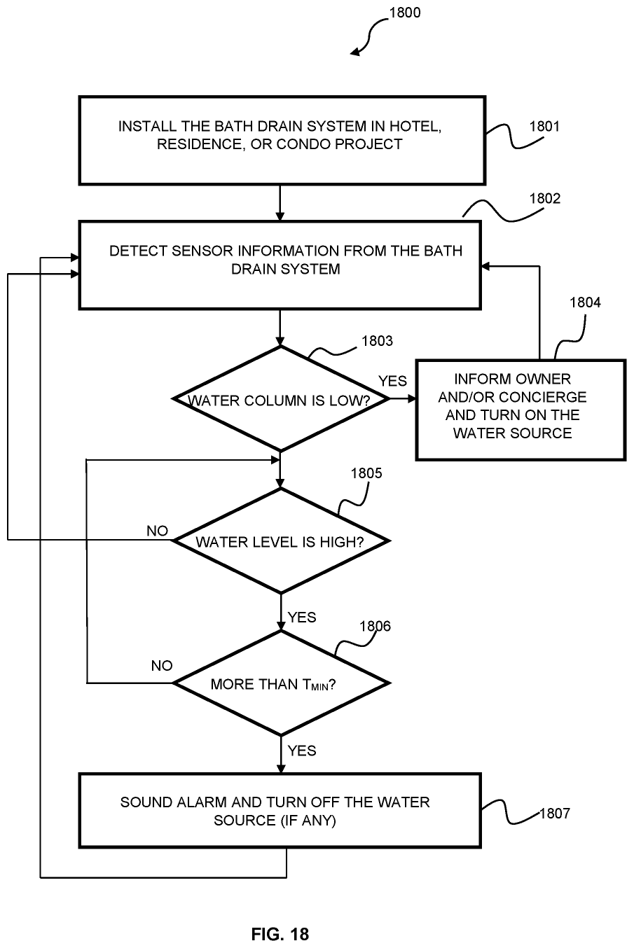

Memory 1570 includes an Operating system (OS) 1571, a basic operating system 1572, a data storage 1580 which includes sensor codes 1581 and data storage 1592. More specifically, memory 1570 stores Basic input/output system (BIOS) 1571 for controlling the internal operations of server 1540. Memory 1470 also stores an operating system (OS) 1571 for controlling basic operations of server 1540. Data storage 1580 illustrates examples of computer-readable storage media as well as computer-readable instructions, data structures, program modules or other household data for storage. Data storage 1580 may be allocated to store data of all bath drain systems 700 or 1100 within a hotel, resort, or a condominium project. In some other embodiments, data storage 1580 may store application specific software programs. It will be appreciated that operating system (OS) and Basic input/output system (BIOS) 1572 may include a general-purpose operating system such as a version of UNIX, or LINUX™, or a specialized operating system such as Microsoft Corporation's Windows® operating system, or the Apple Corporation's IOS® operating system. The operating system may include, or interface with a Java virtual machine module that enables control of hardware components and/or operating system operations via Java application programs. These components are similar to generic computers that need not described in details herein. Server 1540 further includes a sensor module 1590 which further includes water sensor algorithm 1491 and odor sensor algorithm 1592, temperature sensor algorithm 1593, light sensor algorithm 1594 which are in communication with data storage 1580 and microprocessor 1511. These algorithms 1591-1594 are written in Adruino, Linux, or C++—codes. They are down loaded to gateway module 1300 which is configured to receive sensor information and issue appropriate commands that will be described in FIG. 18. In many aspects of the present invention, server 1540-1 to 1540-N may send the information to smartphones 1541-1 to 1541-N for action. It is noted that server 1540 may be used to handle other operations in smart home 1400.

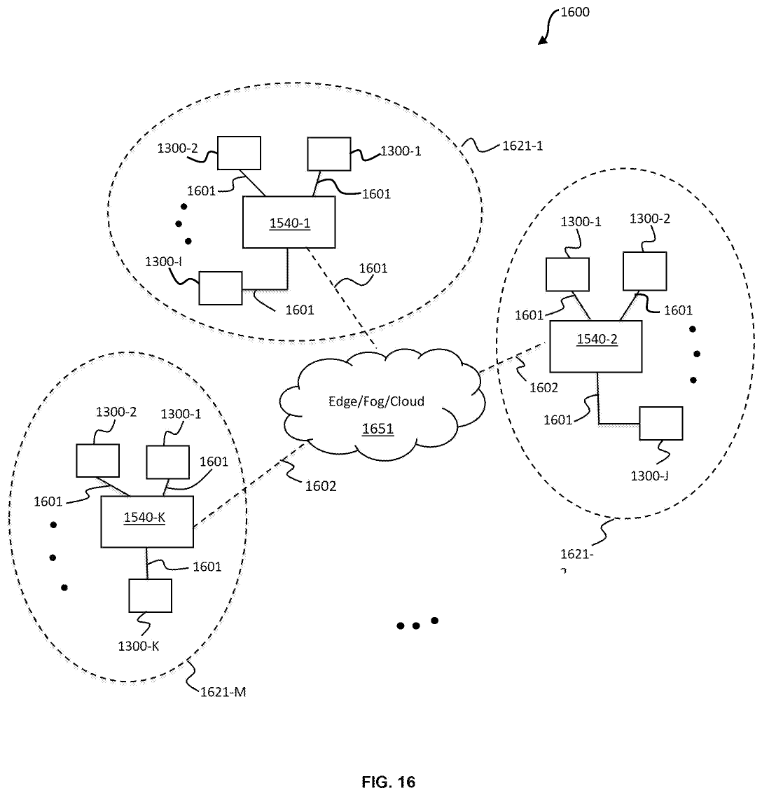

FIG. 16 illustrates a schematic diagram of network of servers 1500 (“network 1500”) that includes a network 1651 connecting servers 1541-1 to 1540-M together which service first integration group 1621-1, second integration group 1621-2, and N^{th} integration group 1621-N of various sensors in accordance with an exemplary embodiment of the present invention. First integration group 1621-1 includes gateway module 1300-1, 1300-2 . . . , 1300-1 connected to server 1540-1 via a local communication channel 1601. Second integration group 1621-2 includes gateway modules 1300-1, 1300-2 . . . , 1300-J connected to a server 1540-2 via local communication channel 1601. N^{th} integration group 1621-M includes gateway modules 1300-1, 1300-2 . . . , 1300-K connected to a server 1540-K via local communication channel 1601. First integration group 1621-1, second integration group 1621-2, and M^{th} integration group 1621-M are connected and communicate to one another to a network 1651 via a network communication channel 1602. In some embodiments of the present invention, network 1651 can be either wireless channels such as can be data center, cloud/edge/fog, or network such as nanonetwork, body area network (BAN), personal area network (PAN), local area network (LAN), campus/corporate area network (CAN), metropolitan area network (MAN), wide area network (WAN), and mesh area networks, edge, cloud, hybrid cloud, or fog network or any combinations thereof. Local communication channels 1601 and network communication channels 1602 can be wireless channels such as Bluetooth, 4G, LTE, 5G, Wi-Fi, Zigbee, Z-wave, radio frequency (RF), Near Field Communication (NFC), Ethernet, LoRaWAN. Alternatively, local communication channels 1601 and network communication channels 1602 can be wired connectors such as RS-232, RS-485, USB, or any combinations thereof, or wired such as RS-232, RS-485, USB, or any combinations thereof.

Continuing with FIG. 16, as non-limiting examples, first integration group 1621-1 can be a different houses (such as 1410) in first tower of a condominium complex. Second integration group 1621-2 can be other houses in a second tower in the same condominium complex. M^{th} integration group 1621-M can be yet other houses in a fifth tower (M=5) of the same condominium complex. In other embodiments of the present invention, first integration group 1621-1 can be a different rooms in a first group of hotel buildings. Second integration group 1621-2 can be other rooms in a second group of hotel buildings. M^{th} integration group 1621-M can be yet rooms in a fifth group of hotel buildings. In different embodiments of the present invention, first integration group 1621-1 can be houses and offices in a first smart city, second integration group 1621-2 can be houses and offices in second smart city, and M^{th} integration group 1621-M are resident houses and/or offices in fourth smart city (where M=4). Gateway modules 1300-1, 1300-2, 1300-I; 1300-1, 1300-2, . . . , 1300-J; and 1300-1, 1300-2, . . . 1300-K include—but not limited to—gateway module 1300 described in FIG. 13 that manages the operations garage door sensors (not shown), humidity sensors (not shown), video cameras (not shown), odor sensors 1331, water level sensors 700 and 1100, and other smart homes sensors and detectors 1333 such as temperature and humidity sensor.

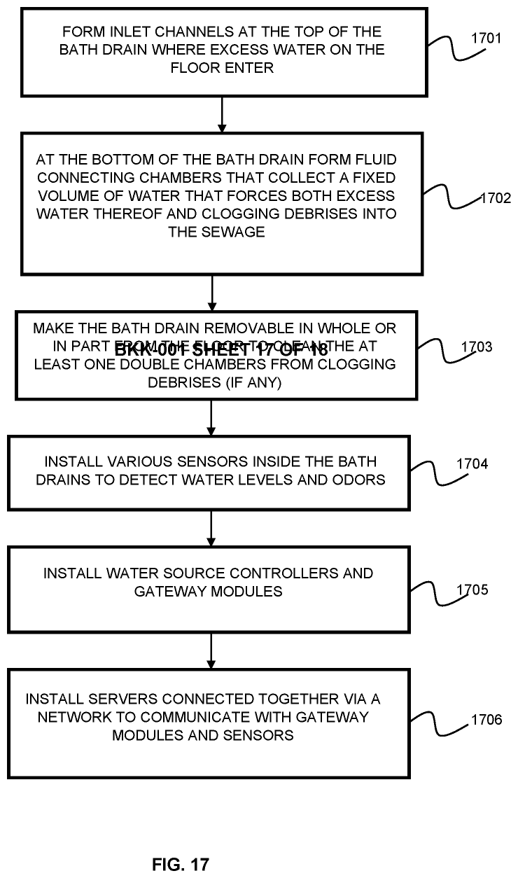

Next referring to FIG. 17, a method 1700 of manufacturing a shower drain that is easy to remove for cleaning and clog-free in accordance with exemplary aspects of the present invention is illustrated.

At step 1701, inlet channels at the top of the brain drain where excess water on the floor enter are constructed. Step 1701 is realized by first type shower drain 200 and second type shower drain 800 described above. In first type shower drain 200, inlet channels are holes 302 (see FIG. 6) forming decorative patterns (see FIG. 3). In many aspects of the present invention, step 1701 is also realized by making bottom side of panel 210 inclined at an angle θ 20° to 30° toward inner core 230. This inclination causes water and debris to drain toward first chamber 601 in fluid connection to second chamber 602 (see FIG. 7). In second type shower drain 800, inlet channels are ditch, gutter, or edge 1101. In many aspects of the present invention, step 1701 is also realized by making bottom side 813 inclined at an angle θ 20° to 30° from all sides toward inner core 910. This inclination causes water and debris to drain toward first chamber 1111 in flood communication with second chamber 1112 (see FIG. 11).

Next at step 1702, forming a water outlet unit including at least two connecting double chambers that collect a fixed volume of water from the inlet channels and overflow both excess water and clogging debrises into the sewer using the connecting tank principle. In many aspects of the present invention, step 1702 is realized by inner core 210 inserted into double layer outer core 230 in first type shower drain 200. Also, step 1702 is realized by inner core 910 inserted into double outer core 930 second type shower drain 800 described above. In first type shower drain 200, the connecting double chambers are hollow space 233 including first chambers 601 in fluid communication with second chamber 602. In second type shower drain 800, connecting double chambers are inside hollow space 833 including first chambers 1111 in fluid communication at the bottom of second chamber. With the connecting double chambers, water and debrises are forced to flow into second chambers 1112 and then to hollow space 1011. Debrises can not settle to the bottom of first chamber 1111 but are forced to flow into second chamber 1112 and then into hollow space 1011 to the sewer due to the forces of water. Please refer to FIG. 7 and FIG. 11 for more details.

At step 1703, shower drain is made removable if there exists a clog. That is, first water inlet module is made removable from bottom water outlet unit. In rare occasions, if there is a puddle of water, remove either the inlet channels and/or the entire shower drain from the floor to clean out collected debris. In first type (surface drain) shower drain 200, step 1703 is carried out by removing lid 300 and inner core 400 using a hook. Alternatively the entire of shower drain 200 can be removed by using a plier gripping at rim 232 and applying a lifting force. In second type of shower drain 800, step 1703 is carried out by unplugging inner panel 810 using a pair of thongs, exposing waste (if any) collected at the bottom of hollow space 833. Alternatively, the entire shower drain 800 is removed to clean or repair by using a plier gripping at rims of first panel 810 and applying a lifting force.

At step 1704, water level sensor circuitry is installed inside the shower drain made from steps 1701-1703 and an odor sensor and other sensors are installed a gateway module. In preferred embodiment of the present invention, water level sensor circuitry is realized by water level sensor system 1200 and gateway module 1300. Odor sensor 1331 is combined inside gateway module 1300 that monitors and manages other sensors such as light sensor 1332, and other sensors 1333.

At step 1705, water source controllers and gateway modules are installed. More specifically, water source controllers communicate with water level sensor level detector and gateway module 1300 so that the turn-on or turn off of water faucets 1434 can be readily controlled. Step 1705 is realized by solenoid valve water faucets available in the market. The solenoid receives command signals from gateway module 1300 or server 1540 to turn on or turn off faucets 1434. In non-limiting examples, when the water level inside chamber 233 and chamber 833 are dry for a preset time period. That is metal probe 1103 is not contacted with water, water level sensor system 1200 sends out a signal to gateway module 1300 and then to server 1540 informing that the water level is low. Gateway module 1300 or server 1540 commands water source controller 1441 to release water 1434 until metal probe 1102 contacts with water. In occasions when the owner forgets to turn off water faucets 1434 or when water tubes 1433 is broken causing a flood in bathroom 1413, water level sensor system 1200 sends out signals informing the owner, concierge, managers about the situation. If the owner forgets to turn off one of the faucets 1426, gateway module 1300 commands water source controller 1441 to turn off that particular faucet.

At step 1706, servers are installed to communicate with sensors and gateway modules via a network. A server can be a desktop computer installed with sensor application module 1590 that includes water sensor algorithm 1591, odor sensor algorithm 1592, temperature sensor 1593, light sensor algorithm 1594, and other sensors such as video/cameras (not shown). An example of water level sensor algorithm 1591 is disclosed in FIG. 18. These algorithms can be Adruino codes, Linux, C++ programing languages. Water level information from water level sensor system 1200 is sent to gateway module 1300 via 433 MHz RF signals by antenna 1224. Antenna 1224 is conveniently placed atop of lid 300 or floor brick 1111 as shown in FIG. 7 and FIG. 11. Upon receiving the water level information, gateway module 1300 reports to server 1540 awaiting for instructions. Server 1540 communicates the water level situation to the owners via smartphones 1412 and/or central control rooms with display panels 1411. After network connection, server 1540 may download various algorithms such as 1591-1593 to gateway modules 1300. Afterward, gateway modules 1300 can manage water sources controllers 1441 and report the data to server 1540 to store them in data storage 1582 for future use.

In summary, the entire method 1700 is realized in practice by first type shower drain 200 and second type (edge drain) shower drain 800 with all features described above.

Finally, referring to FIG. 18, a method 1800 of using water level sensor in accordance with an exemplary aspect of the present invention is illustrated. Method 1800 can be used to control and ensure that shower drain 200 and 800 operate properly.

At step 1801, a bath drain system is installed in bathrooms or shower rooms of a condominium project, a hotel, office building, smart home single houses, or anywhere that needs to drain off excess water. Step 1801 is realized by shower drain 700 and shower drain 1100 equipped with water level sensor system 1200 and antenna 1224. Water level sensor system 1200 is described fully in FIG. 12.

At step 1802, water level and/or flood are detected. Water level information is transmitted to gateway module 1300 located on bathroom wall 1121.

AT step 1803, whether the water level is low is determined. Step 1803 is realized by third metal probe 1103. If water does not make contact with third metal probe 1103, then the water level is low. Water level sensor system 1200 transmits this information to gateway module 1300. Please see FIG. 13.

At step 1804, if the water level is low, then communicating this information to gateway module 1300 or server 1540 which, in turn, informs the owner via smart phones 1412. Either the owner decides to turn on the nearest water faucet 1426 or gateway module 1300 commands water source controllers 1441 to turn on the water.

At step 1805, whether the water level is high is determined. Step 1804 is realized by first metal probe 1101. If water makes contact with first metal probe 1101, then the water level is high. Water level sensor system 1200 transmits this information to gateway module 1300. Please see FIG. 13.

Next, at step 1806 whether the high water level lasts for more than a preset time Tₘᵢₙ. Step 1806 is realized by 555 timer 1322 in gateway module 1300. Tmin can be set by selecting the value of resistors and capacitors connected to trigger pin of 555 timer 1322. The setup of 555 timer 1322 is well-known in the art and described in the data sheet and need not discussed in full here.

At step 1807 if the water level is high for more than preset time Tₘᵢₙ, then communicating this information to gateway module 1300 or server 1540 which, in turn, informs the owner via smart phones 1412. Either the owner decides to turn off water faucet 1426 or gateway module 1300 commands water source controllers 1441 to turn off the water.

In case the water level is high but not for the preset time Tmin, then continuing to monitor the water level. If before Tmin is reached, the water level reduces, then continue to step 1802.

The corresponding structures, materials, acts, and equivalents of all means or step plus function elements in the claims below are intended to include any structure, material, or act for performing the function in combination with other claimed elements as specifically claimed. The description of the present invention has been presented for purposes of illustration and description, but is not intended to be exhaustive or limited to the invention in the form disclosed. Many modifications and variations will be apparent to those of ordinary skill in the art without departing from the scope and spirit of the invention. The embodiment was chosen and described in order to best explain the principles of the invention and the practical application, and to enable others of ordinary skill in the art to understand the invention for various embodiments with various modifications as are suited to the particular use contemplated.

The flow diagrams depicted herein are just one example. There may be many variations to this diagram or the steps (or operations) described therein without departing from the spirit of the invention. For instance, the steps may be performed in a differing order or steps may be added, deleted or modified. All of these variations are considered a part of the claimed invention.

While the preferred embodiment to the invention had been described, it will be understood that those skilled in the art, both now and in the future, may make various improvements and enhancements which fall within the scope of the claims which follow. These claims should be construed to maintain the proper protection for the invention first described.

The foregoing description details certain embodiments of the invention. It will be appreciated, however, that no matter how detailed the foregoing appears in text, the invention can be practiced in many ways. As is also stated above, it should be noted that the use of particular terminology when describing certain features or aspects of the invention should not be taken to imply that the terminology is being re-defined herein to be restricted to including any specific characteristics of the features or aspects of the invention with which that terminology is associated. The scope of the invention should, therefore, be construed in accordance with the appended claims and any equivalents thereof.

### DESCRIPTION OF NUMERALS

100 prior-art shower drain
101 strainer
102 lock nut
103 trap body
104 opening
105 opening
106 inner mesh filter
111 water flow
121 water outflowing direction
200 first type shower drain (surface flow)
220 panel
221 lateral sides of panel with height T₁ and thickness D₁
222 spacers
223 bottom surface of panel
230 double layer outer core
231 outer layer having truncated cone shape
232 inner layer having smaller truncated cone shape
233 reservoir between outer layer and inner layer
234 bottom side of reservoir
240 tailpiece
251 lock nut
300 lid of various drainage patterns
301 solid background
302 openings of lid
300A lotus pattern on lid
301A solid background of lotus flower pattern
302A openings of lotus flower pattern
300B crab pattern on lid
301B solid background of crab pattern
302B openings of crab pattern
303 threaded bolt
400 first type bath (shower) drain
405 hollow space of inner core
406 connector thru hole
410 inner core
411 cylindrical section of inner core
412 bottom side of cylindrical section inner core
413 lateral side of truncated cone section inner core
414 top surface (side) of radius R₁ of truncated cone section
440 water inlet unit
450 water outlet unit
451 lock nut
501 outlet to sewer
600 Assembled first type shower drain
601 first (outer) chamber inside reservoir
602 second (inner) chamber inside reservoir
700 first type shower drain system with sensors
701 water level inside reservoir
711 shower room (bathroom) wall or ceiling.
799 excess water
800 second type (edge flow) shower drain
810 inner (first) panel
811 rim of inner panel
812 bottom side of inner panel
813 brick ejection holes of inner panel
820 outer (second) panel
821 rim of outer panel
822 spacers
823 reservoir mouth
831 outer layer
832 inner layer
833 reservoir
835 reservoir bottom
830 double layer outer core
840 tailpiece
899 edge, or gutter, or ditch
910 inner core
911 lateral side of inner core with radius R₁
912 supporting foot (foot)
913 bottom side of inner core
915 inner hollow space of inner core
940 water inlet unit
940 water outlet unit
1011 outlet to sewer
1100 second type shower drain system with sensors
1101 high metal probe
1102 medium metal probe
1103 low metal probe
1104 electrical wire
1111 first (outer) chamber
1112 second (inner) chamber
1121 bathroom (shower) wall or ceiling
1131 brick
1141 equilibrium water level
1199 running water
1200 water level sensor system
1201 water sensor IC
1210 power supply circuit
1211 battery (voltage supply source)
1212 smoothing circuit
1213 sleep mode circuit
1221 encoder circuit
1222 DIP switch circuit
1223 transmitter circuit
1224 transmitting antenna
1231 electrical wire
1300 gateway module
1300-1 first gateway module
1300-2 second gateway module
1300-I I^{th} gateway module
1300-J J^{th} gateway module
1300-K K^{th} gateway module.
1301 receiving antenna
1302 receiver circuit
1303 DIP switch
1304 decoder
1321 power supply circuit
1322 timer
1331 odor sensor (H₂S)
1332 temperature sensor
1333 other sensors
1341 microcontroller circuit (MCU)
1400 shower room (bath room) equipped with shower drain system
1410 rooms having water pipe
1411 kitchen
1412 laundry room
1413 bathroom
1414 shower room
1421 kitchen sinks
1422 laundry machine
1423 washbasin
1424 toilet bowl
1425 bathtub
1426 faucets
1433 water tubes
1441 solenoid valve water controllers
1442 communication link
1451 display panels
1452 smart phones
1500 server network
1510 network
1511 microprocessor (CPU)
1540 server
1540-1 first server
1540-2 second server
1540-N N^{th} server
1541-1 first smartphone
1541-2 second smartphone
1541-3 N^{th} smartphone
1542 power supply unit
1543 ROM
1544 I/O interface
1545 network interface
1546 audio interface
1551 display
1552 keyboard
1553 pointing device (mouse)
1554 speakers
1570 data storage
1571 operating system (OS)
1572 BIOS
1580 memory banks
1581 Sensor operation codes
1582 memory
1590 sensor application module
1591 water level sensor algorithm
1592 odor sensor algorithm
1583 temperature and humidity sensor algorithm
1594 light sensor algorithm
1595 other sensor algorithm (e.g., camera)
1600 communication network
1601 local communication channels
1602 distant communication channels
1621-1 first integration group (e.g., first tower)
1621-2 second integration group (e.g., second tower)
1621-M M^{th} integration group (e.g., M^{th} tower)
1651 network links (e.g., cloud, edge, fog, hybrid cloud)

What is claimed is:

1. A shower drain for draining off excess water from a floor to a sewer, comprising:
a water inlet unit configured to receive said excess water;
a double layer water outlet unit, mechanically coupled to said water inlet module to form bottom connecting chambers that contain a body of water at a predetermined height operable to force said excess water beyond said predetermined height to overflow into said sewer; wherein said double layer water outlet module further comprises an inner layer concentric with an outer layer that are both open top and close bottoms that form a reservoir; and
a water level sensor system, mechanically installed inside said water inlet unit, operable to sense different water levels inside said reservoir.

2. The shower drain of claim 1, wherein said water inlet module further comprises a lid removably coupled to an inner core configured to mechanically insert between said inner layer and said outer layer to form said bottom connecting chambers.

3. The shower drain of claim 2, wherein said inner core is a truncated hollow cone and wherein said inner layer and said outer layer each is a truncated hollow cone.

4. The shower drain of claim 3, wherein said double layer water outlet unit further comprises:
an outer panel configured to support said lid; and
an outer core further comprising said bottom connecting chambers.

5. The shower drain of claim 4, wherein said outer panel is a first cuboid having an open topside and a solid bottom side.

6. The shower drain of claim 5, wherein said bottom side is inclined at an angle between 20° to 30° toward said double layer outer core.

7. The shower drain of claim 1, wherein said water inlet module further comprises an inner panel connected to an inner core configured to mechanically insert between said inner layer and said outer layer to form said bottom connecting chambers.

8. The shower drain of claim 7, wherein said inner panel is a second open top cuboid having a first width; and wherein said outer panel whose first cuboid having a second width that is larger than said first width.

9. The shower drain of claim 6, wherein said inner layer further comprises a first truncated inverted cone layer with a first diameter and wherein said outer layer further comprises a second truncated inverted cone with a second diameter that is greater than said first diameter; said first truncated inverted cone is concentric with said second truncated inverted cone so that a reservoir is formed there between.

10. The shower drain of claim 7, wherein said first truncated inverted cone has said predetermined height of said body of water.

11. The shower drain of claim 7, wherein said second truncated inverted cone has a second height that is greater than said predetermined height.

12. The shower drain of claim 1, wherein said water level sensor system further comprises:
(a) a water level sensor integrated circuit (IC) operable to sense water levels using a first metal probe, a second metal probe, and a third metal probe placed inside said outlet water unit;
(b) an encoder integrated circuit upon receipt of an output signal from said water level sensor IC assigning a first voltage value for a high water level, a second voltage value for a medium water level, and a third voltage value for a low water level;
(c) a transmitter integrated circuit (IC) operable to modulate said first voltage value, said second voltage value, or said third voltage value to a gateway module for transmission; and
(d) a transmitting antenna, placed on top of said lid, operable to transmit said first voltage value, said second voltage value, or said third voltage value to said gateway module.

13. The shower drain of claim 1, wherein said water level sensor system further comprises a power supply circuit operable to provide voltage supply to said water level sensor IC, said encoder IC, and said transmitter IC; wherein said power supply circuit further comprises a battery, a smoothing circuit, and a sleep mode circuit.

14. The shower drain of claim 1, further comprising a gateway module placed outside of said shower drain for detecting odorous gases originating from said shower drain, a temperature sensor, a light sensor, and a camera.

15. The shower drain of claim 14, wherein said gateway module further comprises:
(a) a receiving antenna for receiving said first voltage value, said second voltage value, or said third voltage value;
(b) a receiver circuit operable to prepare said first voltage value, said second voltage value, or said third voltage value; and
(c) a decoder integrated circuit (IC) operable to translate said first voltage value to said high water level, said second voltage value to said medium water level, and said third voltage value to said lower water level.

16. The shower drain of claim 14, wherein said gateway module further comprises:
(a) a timer, upon receipt of said high water level, operable to count for a threshold time period;
(b) a microcontroller circuit (MCU) operable to determine whether said high water level surpasses said threshold time period; wherein if said high water level surpasses said threshold time period then (i) send an alarm signal to users, and/or (ii) turn off a water source using a solenoid valve controller; and wherein if said low water level occurs then send said alarm signal to said users, and/or (ii) turn on said water source using said solenoid valve controller.

17. A method of fabricating a shower drain, comprising:
(a) forming a water inlet module configured to receive excess water;
(b) forming a double layer water outlet module; and
(c) inserting said water inlet module into said double layer water outlet module to form a reservoir in form of bottom connecting chambers having a predetermined height that force said excess water into a sewer;
(d) providing a water level sensing system inside said double layer water outlet module for sensing water levels inside said reservoir.

18. The method of claim 17 wherein said step (a) of forming a water inlet module further comprises: (e) providing a gateway module that includes a water level receiver integrated circuit, an odor sensor circuit, a temperature sensor circuit, and a camera; wherein said gateway circuit is positioned outside of said shower drain.

19. The method of claim 17 further comprising:
i) detecting a high water level, a medium water level, and a low water level inside said reservoir;
ii) if said high water level is maintained longer than a preset time period, then sending an alarm signal to users and turning off a water source using a solenoid valve water controller in communication with said gateway module; and
iii) if said low water level is achieved, then sending said alarm signal to said users and turning on said water source using said solenoid valve water controller in communication with said gateway module.

20. The method of claim 17 further comprising:
(f) detecting a hydrogen sulfide gas from said shower drain; if said hydrogen sulfide gas is detected then sending said alarm signal to said users and turning on said water source using said solenoid valve water controller in communication with said gateway module.
